(19) United States
(12) Patent Application Publication
(10) Pub. No.: US 2022/0378699 A1
(43) Pub. Date: Dec. 1, 2022

(54) SELF-EMULSIFYING CANNABIS EXTRACT

(71) Applicant: ISRAEL PLASTICS AND RUBBER CENTER LTD., Haifa (IL)

(72) Inventors: Dan LEWITUS, Herzliya (IL); Faran LEVI, Rosh Pina (IL); Rina BINYAMINI, Tel Aviv (IL); Almog UZIEL, Tel Aviv (IL)

(21) Appl. No.: 17/771,578

(22) PCT Filed: Oct. 26, 2020
(86) PCT No.: PCT/IL2020/051115

§ 371 Date: Apr. 25, 2022

Related U.S. Application Data
(60) Provisional application No. 62/986,722, filed on Mar. 8, 2020.
(60) Provisional application No. 62/926,583, filed on Oct. 28, 2019.

(51) Int. Cl. A61K 9/107; A61K 36/185; A61K 9/00; A61K 47/42; A61K 47/40; A61K 47/14; A23L 33/105

(52) U.S. Cl. CPC A61K 9/107; A61K 36/185; A61K 9/0095; A61K 47/42; A61K 47/40; A61K 47/14; A23L 33/105; A23V 2002/00

(57) ABSTRACT

Provided are solid self-emulsifying Cannabis plant extract formulations, methods for their preparation and uses thereof.

### TECHNICAL FIELD

The present disclosure relates generally to solid self-emulsifying Cannabis plant extract, methods for their preparation and uses thereof.

### BACKGROUND

Phytocannabinoids are a family of naturally produced substances that can be found in cannabis plants. The use of cannabis-derived substances as medicinal agents had been shown to be effective against neuropathic pain (Berman et al., 2004), HIV and cancer treatment side effects (Ellis et al., 2009), (Todaro and York, 2012), epilepsy (Sulak et al., 2017), sclerosis, neurogenerative disorders and more (Thompson and Holmes, 2015). The vast majority of cannabinoids clinical research focus on the influence of only two cannabinoids, (—)-trans-Δ⁹-tetrahydrocannabinol (THC) and cannabidiol (CBD), where many terpenoids, flavonoids, and more than 100 different Phytocannabinoids can be found in cannabis plants (Do et al., 2016). Although massive research had been performed on common cannabinoids, the therapeutic effect of the complete composition of a “full spectrum” cannabis medicinal extract (CME) is not fully understood and may lead to novel therapeutic discoveries (Colizzi and Bhattacharyya, 2017). Moreover, Phytocannabinoids composition may vary from one Cannabis strain to another and greatly depend on growth conditions, age, storage and more, thus creating even more therapeutic and research paths (Berman et al., 2018).

The most common delivery strategies for cannabinoids are smoking of dry plant material and oral administration of extracts (Grotenhermen, 2004). Both methods present some advantages and disadvantages. For smoking, some of the advantages are short onset time and high theoretical bioavailability. On the other hand, the action time is very short (1-4 hrs) with high variance in serum concentration. Furthermore, during the combustion of the plant matter, some toxic and carcinogenic compounds are introduced to the mucosal tissue along the inhalation path (“Cyclodextrin-enabled Cannabinoid Formulations”, 2018).

Oral administration of extracts is characterized by lower bioavailability, long onset time, long-acting duration and relatively stable serum concentration when compared to smoking (Grotenhermen, 2004). Some of the disadvantages of oral consumption are low bioavailability, due to poor solubility in water, and decomposition of active compounds along the ingestion path and in the liver (Garrettx and Hunt, 1974).

When relating to oral administration, it is to note that bioavailability depends on the water solubility of the active compounds. According to the Biopharmaceutics classification system (BCS), drugs can be classified according to their absorbance quality. In general, there are two major factors: membrane permeability and water solubility (Fahr and Liu, 2007). The optimal state is achieved when a certain active pharmaceutical ingredient (API) shows both high permeability and high solubility (class 1), and the general target is to shift APIs in class 2,3 and 4 toward class 1. In class 1, high solubility and high permeability lead to good dispersion, and aggregation is prevented. Thus, nearly all API particles are small enough to permeate through the intestine membrane (Pouton, 2006). Class 2 APIs, exhibit water solubility smaller than 1 μg/ml, thus dissolution is the limiting process when relating to absorbance rate and efficiency. It is suggested that alterations in the formulation processes, which may result in an increased dissolution rate or enhanced solubility, may artificially enhance the rate and extent of absorption (Fahr and Liu, 2007).

Emulsified formulations are well-established solubility enhancement techniques in the context of poorly water-soluble solid drugs. Solid substances are usually first dissolved in an oily medium (a carrier phase) and then emulsified in water in the presence of several surfactants (Fahr and Liu, 2007). In that process, the substance becomes amorphous (for crystalline drugs) and dispersed as nano/micro-particles. As a result, an increase in bioavailability and absorption is achieved (Prajapati and Patel, 2007). Emulsified formulations are usually delivered in liquid form, encapsulated in a rigid capsule. After oral intake, the capsule dissolves and the emulsion dilutes in stomach liquids. It is important to mention that although surfactant concentration drops below its critical micelle value during the dissolution process, enhanced absorption is still achieved. On the other hand, this delivery method presents compromised stability and portability, low drug loading, limited dosage options, short shelf life, and most importantly the risk of gastrointestinal irritation due to excessive surfactant content (Prajapati and Patel, 2007; Tang et al., 2008).

An alternative technique known as solid self-emulsifying drug delivery system (S-SEDDS) may provide a solution for the problems associated with liquid emulsion formulations (Tang et al., 2008). For this technique, the most basic approach consists of solidifying a liquid self-emulsifying system to either a solid or a semisolid by absorption to a solid carrier and drying (Tang et al., 2008). Consequently, the resulting product exhibits greater long-term stability but still usually suffers from low drug loading and excessive surfactant content (Nakagawa et al., 2004).

Patent Application Publication No. US 20180007924 is directed to homogenous cannabis compositions and methods of making the same. Patent Application Publication No. US 20170290870 is directed to ingestible films having substances from hemp or cannabis. Patent Application Publication No. US 20180042845 is directed to Preparations of cannabis emulsions and methods thereof.

Nevertheless, there is a need in the art for improved self-emulsifying formulation allowing the storage of whole plant cannabis extract in solid form, while converting it into a homogenous emulsified beverage when mixed with warm liquid, in a wide range of concentrations.

### SUMMARY

Aspects of the disclosure, according to some embodiments thereof, relate to improved self-emulsifying formulations allowing the storage of whole plant cannabis extract (Cannabis medical extract (CME)) in solid form, while turning it into a homogenous emulsified beverage when mixed with warm liquid (such as water) in a wide range of concentrations, which are otherwise hard to obtain.

In some embodiments, the present disclosure provides advantageous compositions, formulations and methods to enhance the solubility of cannabis medical extract (CME), in liquids, for example, for use in beverages, such as, tea preparations, for allowing oral delivery of a desired range of concentrations of various cannabis medical extracts.

In some embodiments, the advantageous compositions, formulations and methods disclosed herein overcome the shortcoming of oral delivery of cannabis extracts in liquid form, which is characterized by lower bioavailability, due to their poor solubility in water, and decomposition of active compounds along the ingestion path and in the liver.

According to some embodiments, the advantageous compositions and formulations disclosed herein are in the form of Solid Self-Emulsifying CME Delivery Systems. The advantageous Solid Self-Emulsifying CME Delivery Systems (S-SECDS) disclosed herein allow the solubilization of cannabis medicinal extract (CME) having high CME loadings (for example, over about 40% w/w) and with food-grade ingredients, facilitating oral delivery/consumption of CME, using, for example, enriched beverages.

In some embodiments, the methods for fabricating/preparing/manufacturing the formulations disclosed herein include preparing a concentrated emulsion followed by gelation and dehydration of the emulsion, to result in dried formulations advantageously capable of being dehydrated at will in liquid, such as, warm or hot water.

According to some embodiments, as exemplified herein, the solid self-emulsifying CME delivery system (S-SECDS) may be assessed for self-emulsifying (SE) properties and bioavailability (in-vivo) thereof. Self-Emulsifying (SE) performances may be characterized by dissolution rate and final emulsion particle size distribution. In some embodiments, as exemplified herein, the obtained solid self-emulsifying CME delivery system exhibited noteworthy high CME loading (over about 40%, for example, over about 50%, (such as, for example, about 56.2% w/w)), while avoiding the use of undesired gastro-intestinal (GI) irritating surfactants. A vast increase (of over 45 fold, for example, 47-fold increase) in dissolution rate and over about 65% (such as about 68.6%) increase in final CME released amount in beverage was found when compared to neat CME. Moreover, emulsion droplet size distribution results demonstrated that use of sonication procedure during the manufacturing/preparation/fabrication process may significantly improve the SE performances of the system.

According to some embodiments, there is provided a solid self-emulsifying Cannabis plant extract formulation comprising whole cannabis plant extact (NICE), a solid carrier and a lipidic component, wherein upon dehydrating said solid formulation in liquid, the cannabis plant extact is self-emulsified within said liquid.

According to some embodiments, the formulation may further include a solubility enhancer.

According to some embodiments, the formulation may further include a powdered form of milk or non-dairy milk and/or of a plant-based protein.

According to some embodiments, the cannabis plant extact may be about 30-60% w/w of the solid formulation.

According to some embodiments, the cannabis plant extact may be about 50-60% w/w of the solid formulation.

According to some embodiments, the cannabis plant extact may be about 30-50% w/w of the solid formulation.

According to some embodiments, the solid carrier may include gelatin, carrageenan, alginate, or combinations thereof.

According to some embodiments, the solid carrier may be about 20-40% w/w of the solid formulation.

According to some embodiments, the solid carrier may be about 25-35% w/w of the solid formulation.

According to some embodiments, the solid carrier may be about 20-30% w/w of the solid formulation.

According to some embodiments, the lipid component may include: tistearin, Glyceryl tistearate, fatty acid, oleic acid, linoleic acid, or any combination thereof.

According to some embodiments, the lipid component may be about 10-50% w/w of the solid formulation.

According to some embodiments, the lipid component may be about lipid component may be about 10-20% w/w of the solid formulation.

According to some embodiments, the lipid component may be about 13-15% w/w of the solid formulation.

According to some embodiments, the solubility enhancer may include a surfactant. In some embodiments, the surfactant may be selected from: Methylated-β-cyclodextrin (m-β-CD), β-cyclodextrin, lecithin, or combinations thereof.

According to some embodiments, the solubility enhancer may be about 2-10% w/w of the solid formulation.

According to some embodiments, the solubility enhancer may be about 1.5-2.5% w/w of the solid formulation.

According to some embodiments, the solubility enhancer may be about 1.7-1.9% w/w of the solid formulation.

According to some embodiments, the powdered form of non-dairy milk may include rice milk, soy milk, almond milk, oat milk, coconut milk hemp milk, or combinations thereof.

According to some embodiments, the powdered form of the plant-based proteins may include soy protein, brown rice protein, pea protein, spirulina protein, hemp seed protein, flaxseed protein, pumpkin seed protein, sunflower seed protein chia seed protein, or combinations thereof.

According to some embodiments, the powdered form of non-dairy milk and/or plant-based protein may be about 2-10% w/w of the solid formulation.

According to some embodiments, the liquid may be warm, at a temperature of over about 50 oC. According to some embodiments, the liquid may be warm, at a temperature of over about 75 oC.

In some embodiments, the liquid is water.

According to some embodiments, the solid self-emulsifying Cannabis plant extract formulation may include whole cannabis plant extract, gelatin, and tristearin, wherein upon dehydrating said solid formulation in liquid, the cannabis plant extract is self-emulsified within said liquid. In some embodiments, such formulation may include about 56% w/w cannabis plant extract, about 28% w/w of gelatin and about 14% w/w of tristearin in a dried, solid formulation. In some embodiments, such formulation may include about 21% w/w cannabis plant extract, about 10.5% w/w of gelatin and about 5% w/w of tristearin in a hydrated formulation, hydrated with about 63% w/w water, to form an emulsion.

According to some embodiments, the loading efficiency of the cannabis plant extract in the formulation may be about 107±2.9% in the dry formulation and about 92±0.9% in the emulsion.

According to some embodiments, the formulation may be for use in the preparation of a beverage.

According to some embodiments, the formulation may be for oral administration or consumption of cannabis extract to a subject.

According to some embodiments, there is provided a method for preparing the solid self-emulsifying Cannabis plant extract formulation, which includes one or more of the steps of:

a) heating the cannabis extract and a lipid or fatty acid in a suitable container to obtain a first mixture;

b) dissolving a solid carrier and emulsifying agent in heated aqueous solution to obtain a second mixture;

c) mixing the first mixture and the second mixture to obtain a mixture,

d) heating and/or mixing the mixture to obtain a homogeneous solution or emulsion; and

e) drying the homogenous solution or emulsion to obtain a solid mixture.

According to some embodiments, the preparation method may further include a step of grinding the solid mixture to solid particles.

According to some embodiments, the first mixture may be heated to a temperature of about 50-100 oC. In some embodiments, the second mixture may be dissolved in water at a temperature of about 40-95 oC.

According to some embodiments, mixing the first and second mixtures may be performed under repeated heating.

According to some embodiments, the homogenous solution or emulsion may be obtained using sonication and/or homogenization.

According to some embodiments drying the homogenous solution or emulsion may include freeze drying or evaporation of the liquid.

According to some embodiments the method may further include a step of grinding the solid mixture to solid particles.

According to some embodiments there is a method of preparing a solid self-emulsifying Cannabis plant extract formulation, the method includes one or more of the steps of:

a) heating cannabis extract and tristearin in a suitable container to obtain mixture;

b) optionally mixing the obtained mixture and adding a solubility enhancer;

c) adding gelatin to the heated mixture;

d) heating and/or mixing the mixture to obtain a homogeneous solution.

In some embodiments, the method may further include a step of sonicating the obtained solution.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1 shows a schematic drawing illustrating mode of action of self-emulsifying (SE) cannabis plant extract particles, according to some embodiments. The SE particles action mechanism include the stages/phases of breakdown (transition from A to B) and dissolution (transition from C to D);

FIGS. 2A-B show pictograms of vials demonstrating optical clarity of the vials containing water (neat water) or CME tea (CME emulsions), in concentrations representing a dose of 0.1mg/kg, 1 mg/kg and 10 mg/kg, immediately (FIG. 2A) and 24 hours (FIG. 2B) after preparation;

FIGS. 3A-B show line graph plots of final emulsions (tea) particle size distribution (class size (μm) vs. volume density (%)): FIG. 3A shows the results of the unsonicated formulation; and FIG. 3B shows the results of the sonicated formulation;

FIGS. 4A-B show microscopic images of dehydrated solid formulation (gel) at X400 magnification of: FIG. 4A unsonicated formulation (Fl), and FIG. 4B-sonicated formulation (F2);

FIGS. 5A-B show snapshot sequence of images (at time points 0, 4, 8, 12, 16 and 20 seconds from stirring) of a video of neat CME and SE particles during the process of beverage preparation. The sequence of images demonstrates the in dissolution rate of SE particles (FIG. 5A) and the neat CME (FIG. 5B);

FIG. 6 shows line graphs of the dissolution profiles over time (s) of neat CME or SE particles to CBD and THC phytocannabinoids; and

FIG. 7 presents bar graphs showing the relative amounts of CBD, CBN, THC and CBC phytocannabinoids in the cannabis extact, in the serum from the experiment group (mice orally administered with F2 beverage formulation, providing 5mg/kg of phytocannabinoids) and in serum from the control group mice (mice orally administered with Cremophor EL (castor oil) based emulsion MCE formulation (providing 5mg/kg of phytocannabinoids).

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, provided herein an advantageous solid self-emulsifying CME delivery system (S-SECDS). In some embodiments, the S-SECDS allows the solubilization of cannabis medicinal extract (CME) having high CME loading capabilities (for example, over about 50% w/w) and with food-grade ingredients, allowing oral delivery consumption of CME enriched beverages, such as, hot beverages.

In some embodiments, the formulation method includes preparing/manufacturing/fabricating a concentrated emulsion, followed by gelation and dehydration of said emulsion, for optionally being self-emulsified when rehydrated.

In some embodiments, the provided delivery system exhibits rapid self-emulsification properties from the solid preparation, as exemplified below.

In some embodiments, the provided system may be used as edible powder, consumed as-is or optionally combined with other foods.

According to some embodiments, in-vivo evaluation show that the self-emulsifying system may be comparable in terms of delivery efficiency and uniformity to liquid-based Cremophor EL emulsions. Moreover, the formulations disclosed herein exhibit solubilizing properties while avoiding the risk of gastrointestinal irritation that is usually associated with potent solubilizes, such as Cremophor EL.

According to some embodiments, the solid self-emulsifying CME delivery system (S-SECDS) may be assessed for self-emulsifying (SE) properties and bioavailability (in-vivo). by determining dissolution rate and final emulsion particle size distribution. As demonstrated herein, the obtained delivery exhibit showed noteworthy high CME loading (of over about 56%%w), while avoiding the use gastro-intestinal (GI) irritating surfactants, such as, Cremophr EL. Further, over about 47-fold increase in dissolution rate and over about 68% increase in final CME released amount were exemplified, when compared to neat CME. Moreover, emulsion droplet size distribution suggested that execution of sonication procedure during the preparation process of the formulation, may significantly improve the self-emulsifying performances of the system.

According to some embodiments, the formulations disclosed herein may further include one or more nutritional or other additives, such as, for example, other plant-based protein (other than CME), non-dairy milk, and the like.

According to some embodiments, a “CME” or “cannabis medicinal extract” relates to extracts directly obtained from a cannabis plant. In some embodiments, CME relates to extracts of the entire/whole cannabis plant. In some embodiments, CME relates to extracts form a cannabis plant which includes a plurality of

Phytocannabinoids. In some embodiments, CME relates to extracts form a cannabis plant which includes the entire repertoire of phytocannabinoids from the plant. In some embodiments, CME relates to extracts form a cannabis plant which includes at least a portion of entire repertoire of phytocannabinoids from the plant.

As used herein, the term “Phytocannabinoids” relate to structurally diverse naturally occurring chemical constituents in the genus Cannabis (Cannabaceae). In some embodiments, phytocannabinoids may include such compounds as: tetrahydrocannabinol, cannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-Ca, cannabidivarinic acid, cannabidiorcol, delta-9-tetrahydrocannabinolic acid A, delta-9-tetrahydrocannabinolic acid B, delta-9-tetrahydro cannabinolic acid-Ca, delta-9-tetrahydrocannabivarinic acid, delta-9-tetrahydrocannabivarin, delta-9-tetrahydrocan nabiorcolic acid, delta-9-tetrahydrocannabiorcol, delta-7 cis-iso-tetrahydrocannabivarin, delta-8-tetrahydrocannabiniolic acid, delta-8—tetrahydrocannabinol, cannabicyclolic acid, cannabicylovarin, cannabielsoic acid A, cannabielsoic acid B, cannabinolic acid, cannabinol methylether, cannabinol-Ca, cannabinol—C, cannabiorcol, 10-ethoxy-9-hydroxy delta-tetrahydrocannabinol, 8,9-dihydroxy-delta-6a-tetrahydrocannabinol, cannabitriolvarin, ethoxy cannabitriolvarin, dehydrocannabifuran, cannabifuran, cannabichromanon, cannabicitran, 10-oxo-delta-6a-tetrahy drocannabinol, delta-9-cis-tetrahydrocannabinol, 3, 4, 5, 6 tetrahydro-7-hydroxy-alpha-alpha-2-trimethyl-9-n-propyl 2, 6-methano-2H-1-benzoxocin-5-methanol-cannabiripsol, and trihydroxy-delta-9-teirahydrocannabinol. In some embodiments, the phytocannabinoid may include tetrahydrocannabinol (THC) and/or cannabidiol (CBD). In some embodiments, the Phytocannabinoids may be selected from: (—)-trans-Δ9-tetrahydrocannabinolic acid (Δ9-THCA), cannabidiolic acid (CBDA), cannabigerolic acid (CBGA), cannabichromenic acid (CBCA), cannabinolic acid (CBNA), cannabidivarinic acid (CBDVA), (—)-trans-Δ9-tetrahydrocannabinol (Δ9-THC), cannabidiol (CBD), cannabigerol (CBG), cannabichromene (CBC), cannabinol (CBN), (—)-trans-Δ9-tetrahydrocannabivarin (Δ9-THCV), cannabidivarin (CBDV), cannabichromevarin (CBCV), Δ9-THC-d3, CBD-d3 and CBN-d3.

According to some embodiments, the solid self-emulsifying Cannabis plant extract formulations disclosed herein may include over about 20% of the cannabis plant extract. In some embodiments, the solid formulations may include over about 30% of the cannabis plant extract. In some embodiments, the solid formulations may include over about 40% of the cannabis plant extract. In some embodiments, the solid formulations may include over about 50% of the cannabis plant extract. In some embodiments, the solid formulations may include over about 55% of the cannabis plant extract. In some embodiments, the solid formulations may include over about 60% of the cannabis plant extract. In some embodiments, the solid formulations may include over about 65% of the cannabis plant extract. In some embodiments, the solid formulations may include over about 70% of the cannabis plant extract. In some embodiments, the solid formulations may include over about 80% of the cannabis plant extract. In some embodiments, the solid formulations may include about 20-80% w/w of the cannabis plant extract. In some embodiments, the solid formulations may include about 25-70% w/w of the cannabis plant extract. In some embodiments, the solid formulations may include about 40-60% w/w of the cannabis plant extract. In some embodiments, the solid formulations may include about 50-60% w/w of the cannabis plant extract. In some embodiments, the solid formulations may include about 55% w/w of the cannabis plant extract. In some embodiments, the solid formulation may include about 56% w/w. In some embodiments, the solid formulations may include about 20-85% w/w of the cannabis plant extract, or any subranges thereof. Each possibility is a separate embodiment.

According to some exemplary embodiments, when the solid particles are dehydrated, the cannabis plant extract may be about 2-60% w/w or w/v of the solution. In some embodiments, when the solid particles are dehydrated, the cannabis plant extract may be about 5-50% w/w or w/v of the solution. In some embodiments, when the solid particles are dehydrated, the cannabis plant extract may be about 10-40% w/w or w/v of the solution. In some embodiments, when the solid particles are dehydrated, the cannabis plant extract may be about 15-30% w/w or w/v of the solution. In some embodiments, when the solid particles are dehydrated, the cannabis plant extract may be about 20-25% w/w or w/v of the solution. In some embodiments, the final concentration of the CME (or components thereof) may depend on the amount of liquid added, temperature, pressure, time of dissolution, and the like, or any combination thereof.

In some embodiments, the amount of CME in the solid formulation or hydrated formulation may be adjusted so as to allow a dosage of CME (or components thereof, such as CBD and/or THC), in the range of 0.1-10 mg/kg body weight, or any subranges thereof.

According to some embodiments, the solid formulation includes a suitable solid carrier. The solid carrier may include, for example, synthetic and natural gums such as tragacanth, acacia, alginate, dextran, sodium carboxymethylcellulose, methylcellulose, polyvinyl-pyrrolidone, carrageenan, gelatin, or any combination thereof. Each possibility is a separate embodiment. In some embodiments, the solid carrier may be selected from gelatin, carrageenan or alginate. In some embodiments, one or more of the solidifying properties of the solid carrier may change under increased temperature. In some embodiments, the solid carrier is solid or substantially solid, semi-solid, hard, semi-hard, gel, or soft at room temperature. Each possibility is a separate embodiment. In some embodiments, the solid formulation may include about 10-50% w/w solid carrier. In some embodiments, the solid formulation may include about 15-40% w/w solid carrier. In some embodiments, the solid formulation may include about 20-30% w/w solid carrier. In some embodiments, the solid formulation may include about 22-28% w/w solid carrier. In some embodiments, the solid formulation may include about 27% w/w solid carrier. In some embodiments, the solid formulation may include at least about 20% w/w solid carrier. In some embodiments, the solid formulation may include at least about 25% w/w solid carrier.

According to some embodiments, the solid formulation further includes a lipid component. The lipid components may include for example, various types of glycerides and/or various types of fatty acids. In some embodiments, various types of glycerides may be selected from, but not limited to: triglycerides, medium-chain tiglyceride, short-chain triglyceride, partial glyceride, glyceryl tristearate, glyceryl stearate, and the like, or any combination thereof. In some embodiments, various types of fatty acids may be selected from, but not limited to: polyoxyethylated fatty alcohol, polyoxyethylated fatty acid, polyoxyethylated fatty acid, ester of fatty acids, vegetable oil, oleic acid, linoleic acid, olive oil, soybean oil, grape seed oil, sunflower oil, peanut oil, corn oil, canola oil, coconut oil, and the like, or any combinations thereof. Each possibility is a separate embodiment. In some embodiments, the solid formulation may include about 2-40% w/ lipid component. In some embodiments, the solid formulation may include about 4-20% w/w lipid component. In some embodiments, the solid formulation may include about 6-18% w/w lipid component. In some embodiments, the solid formulation may include about 8-15% w/w lipid component. In some embodiments, the solid formulation may include about 10-15% w/w lipid component. In some embodiments, the solid formulation may include at least about 10% w/w lipid component. In some embodiments, the solid formulation may include at least about 14% w/w lipid component. In some embodiments, the solid formulation may include over about 10% w/w lipid component. In some embodiments, the solid formulation may include over about 14% w/w lipid component.

According to some embodiments, the formulation may further optionally include a solubility enhancer, such as, a surfactant, wherein the solubility enhancer may be selected from, but not limited to: methyl-β-cyclodexirin, β-cyclodexirin, lecithin, and the like, or any combination thereof. Each possibility is a separate embodiment. According to some embodiments, the solid formulation may include, for example, about 1-10% w/w of solubility enhancer. In some embodiments, the solid formulation may include, for example, about 2-8% w/w of solubility enhancer. In some embodiments, the solid formulation may include, for example, about 3-7% w/w of solubility enhancer. In some embodiments, the solid formulation may include, for example, about 4-6% w/w solubility enhancer. In some embodiments, the solid formulation may include, for example, at least about 2% w/w solubility enhancer. In some embodiments, the solid formulation may include, for example, over about 2% w/w solubility enhancer.

According to some embodiments, the solid formulation may further optionally include a powdered form of milk, non-diary milk and/or plant-based proteins. In some embodiments, the inclusion of a powdered form of milk, non-diary milk and/or plant-based proteins may be in addition to or alternatively to the inclusion of the solubility enhancer. In some embodiments, powdered form of non-dairy milk may be selected from, but not limited to: rice milk, soy milk, almond milk, oat milk, coconut milk, hemp milk, and the like, or any combinations thereof. In some embodiments, plant based protein powders may include proteins derived from plants, selected from, but not limited to: soy, brown rice, pea, spirulina, hemp seeds, flaxseed, pumpkin seeds, sunflower seeds, chia seeds, and the like, or any combination thereof. Each possibility is a separate embodiment. According to some embodiments, the solid formulation may include, for example, about 1-10% w/w of powdered form of non-dairy milk and/or plant-based proteins. In some embodiments, the solid formulation may include, for example, about 2-8% w/w of powdered form of non-dairy milk and/or plant-based proteins. In some embodiments, the solid formulation may include, for example, about 3-7% w/w of powdered form of non-dairy milk and/or plant-based proteins. In some embodiments, the solid formulation may include, for example, about 4-6% w/w of powdered form of non-dairy milk and/or plant-based proteins. In some embodiments, the solid formulation may include, for example, at least about 2% w/w of powdered form of non-dairy milk and/or plant-based proteins. In some embodiments, the solid formulation may include, for example, over about 2% w/w of powdered form of non-dairy milk and/or plant-based proteins.

According to some embodiments, there is provided a solid self-emulsifying Cannabis plant extract formulation which includes one or more of: (I) a whole-plant cannabis extract; (II) a solid carrier, including, for example, gelatin, carrageenan or alginate, (III) a lipid component, including, for example, triglycerides (such as glyceryl tristearate (GT)) and/or fatty acids (oleic acid, linoleic acid); (IV) a surfactant, such as methyl-β-cyclodextrin, β-cyclodextrin or lecithin and/ or (V) a powdered form of non-dairy milk (including, for example, rice milk, soy milk, almond milk, oat milk, coconut milk and/or hemp milk) and/or plant-based proteins (such as, soy, brown rice, pea, spirulina, hemp seeds, flaxseed, pumpkin seeds, sunflower seeds and/or chia seeds). Each possibility is a separate embodiment. In some embodiments, the disclosed formulation may include at least about 40% w/w of (I), about 20-30% w/w of (II), about 10-15% w/w of (III), about 2-10% w/w of (IV) and/or about 2-10% w/w of (V).

According to some embodiments, there is provided a solid self-emulsifying Cannabis plant extract formulation which includes: (I) a whole-plant cannabis extract; (II) a solid carrier, (III) and a lipid component. In some embodiments, the formulation may further include: (IV) a surfactant, and/ or (V) a powdered form of non-dairy milk and/or plant-based proteins

According to some embodiments, there is provided a solid self-emulsifying Cannabis plant extract formulation which includes: (I) a whole-plant cannabis extract; (II) a solid carrier, (III) a lipid component and: (IV) a surfactant, and/ or (V) a powdered form of non-dairy milk and/or plant-based proteins.

According to some embodiments, there is provided a solid self-emulsifying Cannabis plant extract formulation which includes: (I) a whole-plant cannabis extract; (II) a solid carrier, (III) a lipid component and (IV) a surfactant.

According to some embodiments, there is provided a solid self-emulsifying Cannabis plant extract formulation which includes: (I) a whole-plant cannabis extract; (II) a solid carrier, (III) a lipid component and (V) a powdered form of non-dairy milk and/or plant-based proteins.

In some embodiments, the disclosed formulations may include at least about 40% w/w of (I) (for example, about 55% w/w), about 20-30% w/w of (II) (for example, about 27% w/w), about 10-15% w/w of (III) (for example, about 14% w/w); and about 2-10% w/w of (IV) (for example, about 2.7% w/w) and/or about 2-10% w/w of (V) (for example, about 2.7% w/w).

In some embodiments, the solid formulation is solid, substantially solid, hard, semi-hard, soft, semi-soft at room temperature. In some embodiments, the solid formulation is not liquid at room temperature.

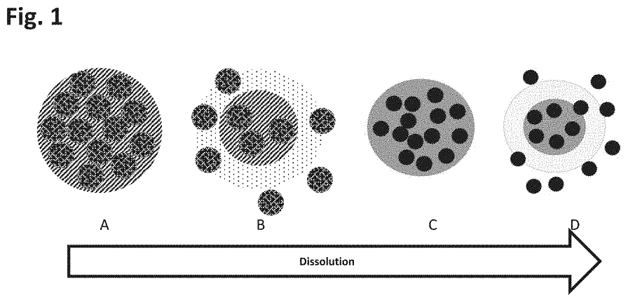

According to some embodiments, without wishing to be bound to any theory or mechanism, the morphological structural changes (dissolution) of the delivery system may include two phases: an oily phase, which includes a lipid component (such as GT) and the CME, encapsulated inside a dehydrated solid carrier matrix (such as, gelatin). Small particles are then released to the liquid media from the surface of the formed particles (which may be spheres), as the hot liquid dissolves the solid carrier matrix. Reference is made to FIG. 1, which shows a schematic illustation of the morphological changes of the solid carrier system: in the first stage/phase transition from state A to state B occurs, which includes the breakdown of whole particles (shown as spheres) to smaller pieces/particles. The second stage occurs in the transition from state C to state D, which includes the release of oily phase droplets from the surface of the particles to the surrounding medium (liquid), to thereby result in enhanced dissolution of the CME in the liquid, as exemplified herein.

In some embodiments, upon dehydrating the solid formulation in liquid, the cannabis plant extract is self-emulsified within the liquid, which may be an aqueous liquid, such as, water. In some embodiments, the liquid is a warm liquid. In some embodiments, the liquid may further include one or more flavorants (natural or artificial). In some embodiments, the temperature of the liquid is over about 35 oC, 40 oC, over about 50 oC, over about 60 oC, over about 70 oC, over about 75 oC, over about 80 oC, or over about 90 oC.

In some embodiments, upon dissolution or dehydrating of the solid formulation in liquid, a beverage is formed, wherein the beverage may be orally consumed. In some embodiments, the beverage is a type of a tea. In some embodiments, the resulting beverage is tea.

In some embodiments, upon dissolution/dehydration of the solid formulation in liquid, the CME or components thereof are dissolved in the liquid and are bioavailable by oral consumption.

In some embodiments, as exemplified herein, after oral consumption of the CME hydrated formulations, the ratio between the cannabis extract phytocannabinoids components (such as, CBD, CBN, THC and CBC) found in blood serum samples is maintained as compared to their ratios in the CME extract, indicating that the oral formulation allows for uniformity in the uptake of the various components as well as the improved oral bioavailability thereof.

According to some embodiments, there is provided a method of preparing a solid self-emulsifying Cannabis plant extract formulation, the method includes one or more of the steps of:

heating (for example, to a temperature of about 50-100 oC, such as, 80 oC) the cannabis extract and a lipid component (such as, glyceride, lipid or fatty acid) in a suitable container to obtain a first mixture;

dissolving a solid carrier and emulsifying agent in heated water (for example, to a temperature of about 40-95 oC), under constant stirring to obtain a second mixture;

mixing the first mixture and the second mixture, heating and/or mixing the mixture to obtain a homogeneous solution or emulsion;

drying the homogenous mixture (solution or emulsion) to obtain a solid mixture; and

optionally, grinding the solid mixture to solid particles (having a desired size, shape and/or form).

In some embodiments, the method of preparation may further include a step of sonicating or homogenizing the obtained solution.

According to some embodiments, there is provided a solid self-emulsifying Cannabis plant extract formulation which includes whole cannabis plant extract, gelatin, and tristearin, wherein upon dehydrating the solid formulation in liquid, the cannabis plant extract is self-emulsified within said liquid. In some embodiments, the formulation may further include a solubility enhancer.

According to some embodiments, the formulation is for the preparation of a beverage. In some embodiments, at least a portion of the beverage is aqueous

According to some embodiments, the formulation is for oral administration of cannabis extract (such as whole plant cannabis extract) to a subject.

According to some embodiments, there is provided a method of preparing a solid self-emulsifying Cannabis plant extract formulation, the method includes the steps of:

heating cannabis extract and tristearin in a suitable container to obtain mixture;

optionally mixing the obtained mixture and adding a solubility enhancer;

adding gelatin to the heated mixture;

heating and/or mixing the mixture to obtain a homogeneous solution.

In some embodiments, the method of preparation further includes a step of sonicating the obtained solution.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Materials and Methods

### Materials

Cannabis extracts were provided by the “Technion—Israel Institute of Technology” biology department. Gelatin (G 2625 from porcine skin), methylated-β-cyclodextrin (33265) (m-β-cd), phosphate-buffered saline (PBS) (D8537-1) and glyceryl tristearate (69498-250G-F) (GT) were all purchased from Sigma Aldrich®.

Ethanol (EtOH), methanol (MeOH), acetonitrile (ACN), acetic acid and water (HPLC grade) were purchased from Biolab®. Cremophor EL® (238470) was purchased from Cal-biochem®. (—)-trans-Δ⁹-tetrahydrocannabinolic acid (Δ⁹-THCA), cannabidiolic acid (CBDA), cannabigerolic acid (CBGA), cannabichromenic acid (CBCA), cannabinolic acid (CBNA), cannabidivarinic acid (CBDVA), (—)-trans-Δ⁹-tetrahydrocannabinol (Δ9-THC), cannabidiol (CBD), cannabigerol (CBG), cannabichromene (CBC), cannabinol (CBN), (—)-trans-Δ⁹-tetrahydrocannabivarin (Δ⁹-THCV), cannabidivarin (CBDV), cannabichromevarin (CBCV), Δ⁹-THC-d3, CBD-d3 and CBN-d3 were purchased from Sigma-Eldrich (Rehovot, Israel). All the phytocannabinoid standards were of analytical grade (>98%).

Cannabinoids quantitation by high-performance liquid chromatography (HPLC).

In some instances, HPLC analysis was carried out using a UHPLC system (Thermo Scientific, Bremen, Germany) fitted with a C18 core-shell column (2.6 μm, 150 mm×2.1 mm i.d.) and a SecurityGuard Ultra column (2 mm×2.1 mm i.d) (Phenomenex, Torrance, Calif., USA). Chromatographic separation was achieved based on a previously described method (Berman et al., 2018) with some adjustments. Component separation was done with a solvent mixture of ACN 0.1%v/v acetic acid (solvent A), water 0.1%v/v acetic acid (solvent B) and MeOH. Solvent gradient was preformed according to the following protocol. 0-4 minuets—constant composition of 50% solvent A, 45% solvent B, 5% MeOH. 4-6 minutes—composition gradually changed to 67% solvent A, 28% solvent B, 5% MeOH. 6-10 minutes—constant composition of 67% solvent A, 28% solvent B, 5% MeOH. 10-14 minuets—composition gradually changed to 90% solvent A, 5% solvent B, 5% MeOH. 14-19 minutes—constant composition of 90% solvent A, 5% solvent B, 5% MeOH. 19-20 minutes—composition gradually changed to 50% solvent A, 45% solvent B, 5% MeOH.

20-25 minutes (column re-equilibration)—constant composition of 50% solvent A, 45% solvent B, 5% MeOH. The column temperature was 30 ° C. and the injection volume was Quantitation was done by a diode-array detector (Diode RA, UltiMate® 3000, Thermoscientific®) at a wavelength of 208 nm. Calibration curves were performed in ethanol for standard mixes of Δ⁹ -THCA, Δ⁹ -THC, CBDA, CBD, CBG, CBGA, CBC,

CBN and CBDV and for the obtained Cannabis extract, over the range of 200 μg/ml to 20 μg/ml.

### CME Loading Efficiency

CME loading efficiency (CLE) was calculated according to the formula in Equation 1.

Equation 1—CLE calculation:

$$CLE = \frac{Actual CME content}{Theoretical CME content} \times 100$$

Actual CME content was obtained by the following process: dry SSEDDS particles were placed in ethanol for 24 hrs. At room temperature under gentle stirring. The ethanol-CME solution was then tested for its CME Content using an HPLC system as described above.

Cannabis-loaded particles (approximately 4 mg) were dissolved in 400 μl hot water (80° C.), then precipitated with 7.6 ml ethanol followed by vortex for 1 h. The suspension was filtered through 0.22 μm PTFE-filters to remove polymeric debris and analyzed for cannabinoid concentration by LC/MS. Cannabinoid loading efficiency was determined by dividing the measured cannabinoid content by the theoretical value.

### Dissolution Rate

In order to evaluate the dissolution properties of the current formulation technique, SE particles were tested against neat CME. The test simulated the process of tea preparation. A test tube, containing CME or SE particles, was placed in a hot water bath (80° C.). Hot di-ionized water and a magnetic stirrer were then added to the test tube. After that, gentle stirring was turned on for 10, 30, 60 or 180 seconds. Three samples were prepared for each stirring period.

At the end of the stirring period, a small sample was taken and diluted in ethanol in a ratio of 1:9 (tea: ethanol). The samples were then left on an orbital shaker for 24 hr.

During that period glyceryl tristearate (GT), gelatin and methylated-β-cyclodextrin (m-β-CD) had fallen out of the solution. The final solutions were then tested for their CME content using an HPLC system (as described above).

### Solubility Test:

Cannabis emulsions were prepared by pouring hot tap water (80° C.) over dry particles. To determine cannabinoid content in the emulsion, 1 ml of supernatant was withdrawn at different time points (from several minutes to a few hours). The obtained supernatant was freeze-dried, dissolved in 1 ml ethanol, filtered, and then analyzed for cannabinoid content by LC/MS.

### Emulsion Droplet Size Characterization

The self-emulsification performances of the different formulations were evaluated by the final emulsion's droplet size distribution. A sample of 20 mg of S-SECDS particles was added to a glass vial. Thereafter, 20 ml of hot (80° C.) tap water were added to the vial and the mixture was gently stirred until complete dissolution was achieved.

The emulsions were then tested for their droplet size distibution using a laser diffraction particle size analyzer (Mastersizer® 3000, Malven®). A refractive index of 1.4385 was chosen as a representative value (refractive index of GT).

### Optical Microscopy

Optical evaluation of the dehydrated gels was performed by drying a small amount of gel between two glass slides. The samples were then tested using bright-field microscopy (OLYMPUS® C10(53). The video, illustating the self-emulsification process was recorded using a microscopic video camera (Dino-lite® RK-10A).

### Final Emulsion Clarity

Due to cannabis use sociological association, for both medicinal and recreational applications, concealment plays a big role in the field of cannabinoid enriched products (Hammersley et al., 2001). For orally-administered cannabinoids, dosage (for THC or CBD) may drastically vary from patient to patient, usually in the range of 0.1-10 mg/kg (Beal et al., 1995; Hill, 2015; Holdcroft et al., 2006; Strasser et al., 2014; Zuardi et al., 2006).

Final emulsions were prepared by pouring hot tap water (80° c) over dry SE particles. The mixtures were then gently agitated until complete dissolvement was observed. Three different concentration were prepared, representing the common dosage spectrum. The concentrations were calculated for a final emulsion (tea) intake of 250 ml, a consumer weight of 70 kg and a CBD content of 41%.

In-Vivo Evaluation

Animal studies were performed in compliance with the Guide for the Care and Use of Laboratory Animals established by the National Institutes of Health. All procedures and protocols were approved by the Technion Administrative Panel of Laboratory Animal Care (#:IL_050-05-2018).

Subjects 10 adult male mice (C57BL/6 J; The Jackson Laboratory), weighing 20-30 gr were housed in standard laboratory conditions with controlled temperature, humidity, light/dark cycle, ad libitum access to water and regular diet. The experiment was performed at noon, during the light cycle.

Preparation and Administration

CME tea was prepared by pouring 13.3 ml of hot di-ionized water (80° c) over 20 mg of dry SE particles, resulting in CME concentration of 1.1mg/ml. The mixture was then genteelly stirred until complete dissolution was observed.

The control solution was prepared by dissolving CME in a solution of 1:1:18 EtOH: Cremophor: PBS. First, 8.5 mg of CME were dissolved in 0.5 ml of EtOH. After that 0.5 ml of Cremophor was added and the mixture was stirred for one hour. Finally, 9 ml of PBS were added, and the mixture was stirred for 30 minutes. Both CME tea and control solution concentrations were calculated to match a dose of 5 mg CBD/kg for a volume intake of 200 μl and an average mouse weight of 25 gr.

For both the control group (5 mice) and the experiment group (5 mice) a dose of 200 μl was orally administered via direct injection to the stomach.

### Blood Collection and Quantitation of Cannabinoids

The mice were sacrificed 1.5 hours after oral administration. Blood samples were immediately taken and stored for further examination.

A sample of 600 μl of an extraction solution containing 0.1% v/v acetic acid in a methanol: acetonitrile 1:1 mixture, spiked with 20 ng/ml deuterated internal standards (ISs) was added to 200 μl serum sample. Samples were than vortexed and centrifuged in order to enable protein and cell precipitation. 3 ml of 0.1% v/v acetic acid in water was then added to the supernatants and the sample was loaded onto Agela Cleanert C8 solid phase extraction (SPE) cartridges (500 mg of sorbent, 50 μm particle size). Elution of phytocannabinoids from the columns was done using 2 ml of 0.1% v/v acetic acid in methanol, followed by an evaporation and reconstituted in 100 μl ethanol.

Cannabinoid quantitation was performed as described by P. Berman et al. (Berman et al., 2018).

### Statistical Analysis

In CME loading efficiency, emulsion droplet size characterization and dissolution rate data were determined as mean ±SD. In the in vivo study, data is presented as mean ±SEM. Significant difference in latency between experimental groups was determined using a paired student t-test with P value <0.05.

### Example 1

### Preparation of SEDDS

In order to evaluate the effect of different preparation methods, two formulations (F1 and F2) were prepared according to the ratios in table 1.

TABLE 1
Formulation ingredients and quantities.

| | Amount | Wet | Dry (theoretical) |
|---|---|---|---|
| | in batch | composition | composition |
| Ingredient | (mg) | % w | % w |
| Active | | | |
| Cannabis extract | 500 | 21 | 56.2 |
| (2532) | | | |
| Matrix/Emulsifier | | | |
| Gelatin | 250 | 10.5 | 28 |
| Carrier phase | | | |
| Glyceryl tristearate | 125 | 5.2 | 14 |
| (GT) | | | |
| Solubility enhancer | | | |
| Methylated-β- | 25 | 0.6 | 1.8 |
| cyclodextrin (m-β-CD) | | | |
| Dispersing phase | | | |
| Water | 1500 | 62.7 | |
| TOTAL | 3800 | 100 | 100 |

F1 was prepared by heating an Eppendorf containing CME and GT in a hot water bath (80° c) for several minutes. After the GT was completely melted, the mixture was vortexed for 30 seconds and then placed back in the water bath. Hot water (80° c) and m-β-CD were added to the Eppendorf and the mixture was vortexed again for 30 seconds and placed back in the water bath. Finally, gelatin was added, and the mixture was vortexed and heated repeatedly until homogenization was observed.

F2 was prepared in two stages, the first stage was the same as the preparation of F1. In the second stage, the mixture was sonicated using a probe sonicator (Qsonica CL 334) fitted with a micro-tip for 60 seconds (Amp=60, lsec on lsec off intervals). For all formulations, a brown, milky gel was obtained at the end of the process.

After the preparation process, the formulations, while still hot, were transferred to a pneumatic droplet dispensing system (Liquidyn® dispensing system T=40c P1=P2=2bar) (Shpigel et al., 2018). In order to obtain spherical particles, the heated gel was printed on aluminum slides that were previously coated with a super hydrophobic coating. The droplets were then left to dry at room temperature for 30 minutes. For further drying, the particles were placed in a vacuum oven (25° c) for 24 hrs.

Other formulation prepared included 48.8 gr extract (30.8 before drying), Gelatin 24.4 (15.4 before drying); M-b-CD 2.4 (1.5 before drying); water 0 (36.9 before drying; either margarine 24.4 (15.4 before drying) or tristearin 24.4 (15.4 before drying). These formulations were prepared as F1 above. These formulations did not provide uniform particle size and the emulsion was not uniform, as fat particles or droplets were observed.

Formulations F1 and F2 were used in some of the further studies below.

### Example 2

### Formulations of Whole-Plant Cannabis Extracts with additional Additives

Formulation I: Whole-plant cannabis extract (100 mg) and glyceryl tristearate (25 mg) were placed in a closed vial and heated in a water bath at 80° C. until complete melting of the glyceryl tristearate. In a different vial, k-carrageenan (50 mg) and rice milk powder (5 mg) were dissolved in hot water (80 C, 10 ml) under constant stiffing. When the components dissolved completely the solution was added to the first vial and the mixture was vortexed and heated repeatedly, then sonicated for 2 min using a probe sonicator to obtain a homogenous emulsion. The water was then removed by freeze-drying and the solid mixture was grinded to small particles.

Formulation II—Whole-plant cannabis extract (100 mg), oleic acid (12.5 mg) and linoleic acid (12.5 mg) were placed in a closed vial and heated in a water bath at 80 C. In a different vial, gelatin (50 mg) and methyl-beta-cyclodextrin (5 mg) were dissolved in hot water (80 C, 4 ml) under constant stirring. When the components dissolved completely the solution was added to the first vial and the mixture was vortexed and heated repeatedly, then sonicated for 2 min using a probe sonicator to obtain a homogenous emulsion. The water was then removed by freeze-drying and the solid mixture was grinded to small particles.

Rice milk powder preparation: Rice milk was made by soaking rice in boiling water (ratio of 1:4 rice to water) for 2 h. Then the rice particles were filtered out and the emulsion was freeze-dried until a fine powder was obtained.

### Example 3

### Alginate-Based Edible Cannabis Formulation

Preparation: Whole-plant Cannabis extract (250 mg) and glyceryl tristearate (62.5 mg) were placed in a closed vial and heated in a water bath at 80 ° C. until complete melting of the glyceryl tristearate. Then, hot water (80 ° C., (15 ml) and Methylated-β-cyclodextrin (12.5 mg) were added to the vial and the mixture was vortexed for 30 sec and placed back in the water bath. At a different vial, sodium alginate (125 mg) was dissolved in 20 ml of hot water (80 cC) under constant stirring. When it dissolves completely, the alginate solution was added to the other vial and the mixture was vortexed and heated repeatedly. Then, the mixture was sonicated for 1 min using probe sonicator, and a brown emulsion was obtained. The emulsion was poured into glass dishes (while still hot) and left to dry overnight at room temperature.

### Example 4

### CME Loading Efficiency

CME loading efficiency (CLE) was tested for both dry particles and final emulsion. CLE of 107±2.9% was found for the dry particles and a CLE of 92±0.9% was achieved for the final emulsion. The variation in CLE between the dry particles and the final emulsion may be attributed to incomplete dissolution of some particles that may be caused by the adherence of semi-dissolved particles to the top of the vial.

### Example 5

### Optical Clarity

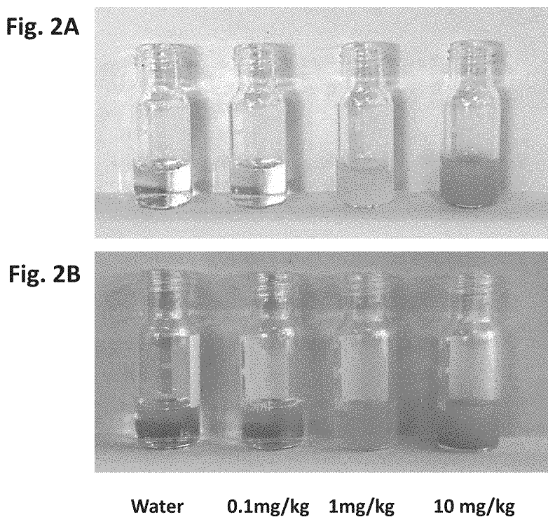

Final emulsions, representing a dosage of 0.1, 1 and 10 mg/kg (MCE) were prepared and optically assessed in comparison to water (FIGS. 2A-B) at time=0 (FIG. 2A) and after 24hrs (FIG. 2B). For the 0.1 mg/kg sample, no change in color was observed but it appeared to be merely cloudier when compared to water. For the 1 mg/kg and the 10 mg/kg samples, a substantial change in color and opacity was observed (FIG. 2A). After 24hrs at room temperature, the emulsions were assessed again in order to evaluate their stability. For the 0.1 mg/kg sample, no change was observed and for the 1 mg/kg and the 10 mg/kg samples, a small amount of sediments was found at the bottom of the vials (FIG. 2B).

### Example 6

### Emulsion Particle Size

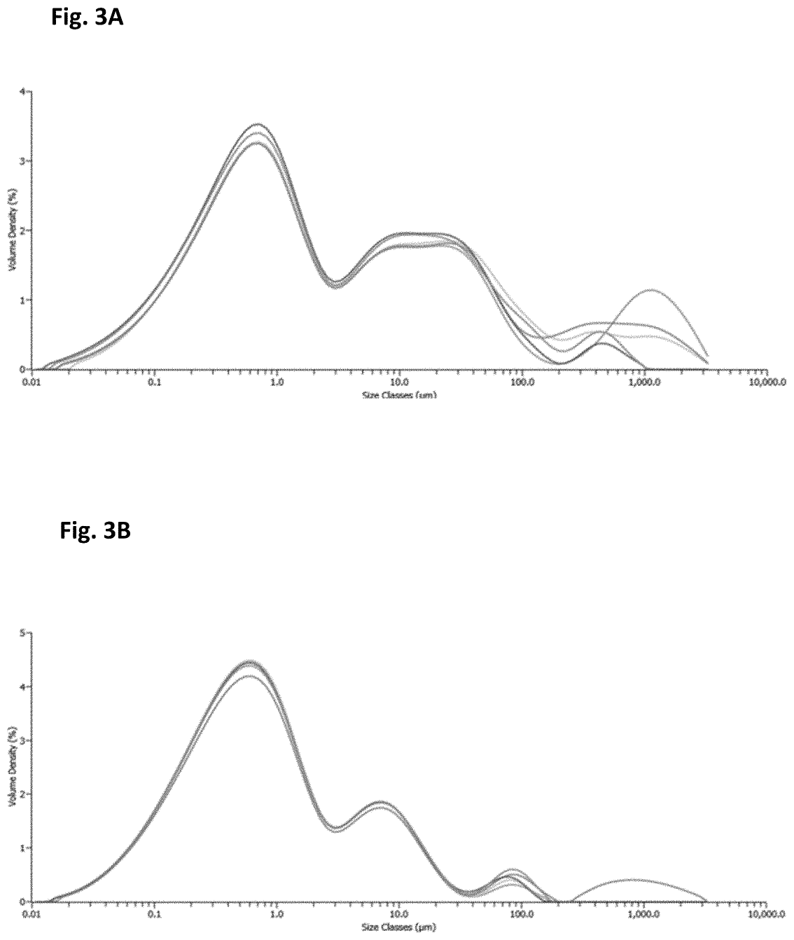

The final emulsions (tea) particle size distribution plots for the different formulation methods are presented in FIGS. 3A-B, where FIG. 3A shows the plots for unsonicated formulation (A) and FIG. 3B shows the plots for sonicated formulation (B). Mean particle size values are shown in Table 2. The size distribution plots were obtained in order to determine whether the sonication process could improve the delivery system's performances. In general, the goal is to achieve particles that are as small and uniform as possible.

A significant improvement in particle size and particle uniformity was observed for F2 (FIG. 3B) over F1 (FIG. 3A), as for example Dx90 was reduced from 193±218 to 10.7±3.2. This observation may be explained by the Gibbs-Marangoni effect. A size reduction of a certain emulsion droplet occurs as a result of the sonication energy input. In the process of size reduction shear forces splits a droplet into two smaller droplets. After the separation, surfactant molecules coat the newly produced surface area thus droplet reattachment is prevented (Singh et al., 2017).

The Dx10, Dx50, and Dx90 relate to percentage of particles having a size value. Dx50 (median), relates to a diameter value where half of the particle's population is below this value. Likewise, 90 percent of the distribution lies below the Dx90, and 10 percent of the population lies below the D×10.

TABLE 2
Mean particle size values for F1
(not sonicated) and F2 (sonicated)

| | F1 | F2 |
|---|---|---|
| Dx10 | 0.18 ± 0.02 | 0.12 ± 0.003 |
| Dx50 | 1.7 ± 0.32 | 0.698 ± 0.033 |
| Dx90 | 193 ± 218 | 10.7 ± 3.2 |

In general, this process will occur until an equilibrium between size reduction and coalescence is achieved. As the energy input of the sonicator is significantly larger than that of the vortex mixer, the system could reach closer to its particle size equilibrium point. Moreover, when looking at the particle size plots, a significant shift toward the main peak can be observed for F2.

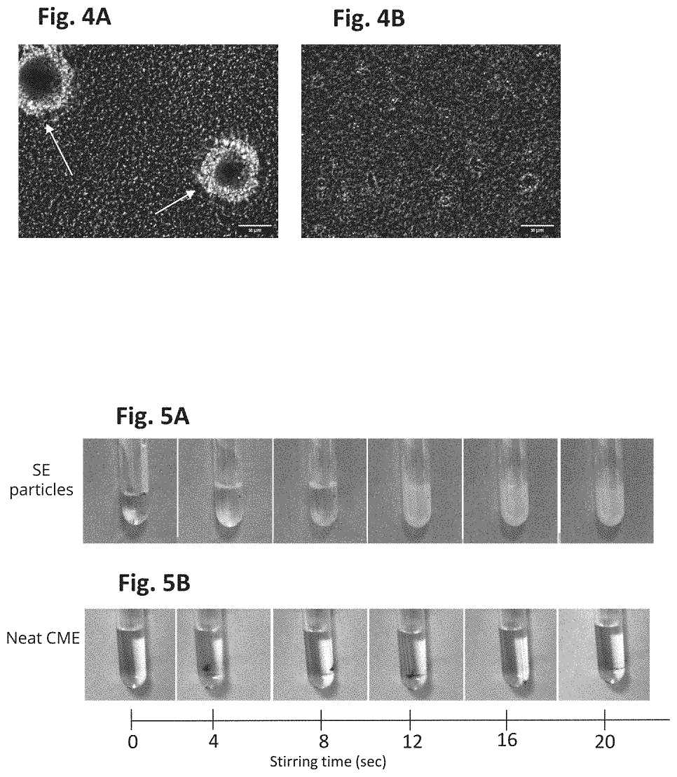

Microscopic images of the dehydrated gels are presented in FIGS. 4A-B. Shown in FIG. 4A are microscopic images of unsonicated gel (F1) and FIG. 4B shows microscopic images of sonicated gels (F2). The results shown in FIGS. 4A-B demonstrates the size reduction process between the two formulations, in particular, reduction in general particle size and the disappearance of irregularly big particles (marked by white arrows in FIG. 4A) are observed with F2 as compared to Fl.

### Example 7

### Dissolution Rate of Solid Formulations

Snapshot sequence of a video (at time points 0, 4, 8, 12, 16 and 20 seconds from stirring), of neat CME and SE particles in the process of tea preparation is shown in FIGS. 5A-B. The sequence of images demonstrates the differences in dissolution rate between the SE particles (FIG. 5A) and the neat CME (FIG. 5B). For the SE particles, a complete disintegration was observed, and the final tea was obtained as a homogenous emulsion. In contrast, the neat CME adhered to the walls of the glass tube and stayed intact as one lump.

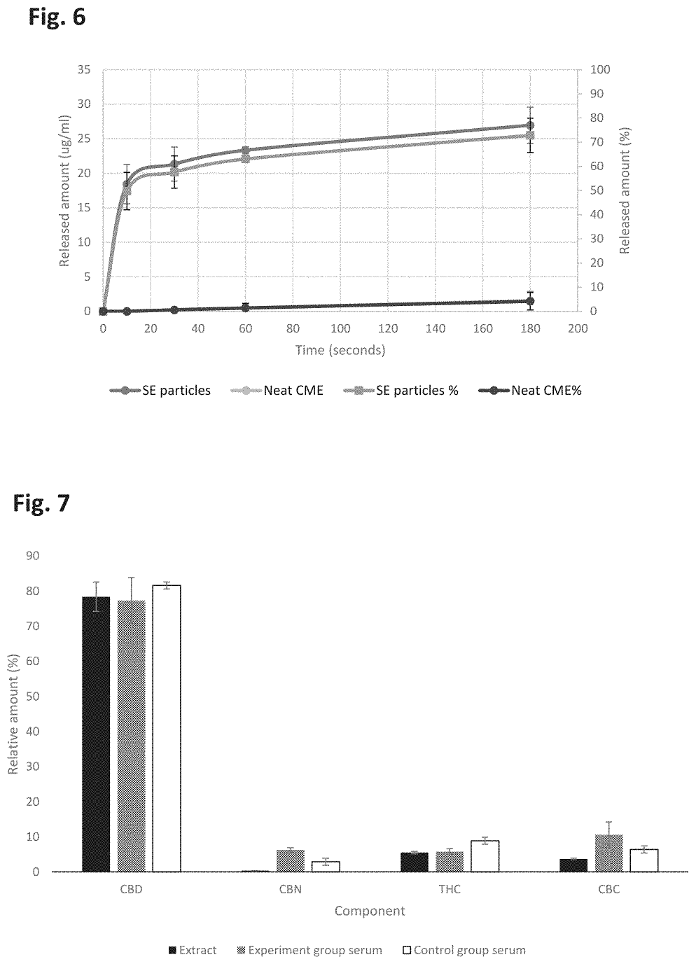

Dissolution profiles of neat CME and SE particles are presented in the graphs shown in FIG. 6. A significant improvement in dissolution rate, final released amount and cannabinoids (CBD and THC) release uniformity was observed for the SE particles when compared to neat CME. For the first ten seconds of stirring, an average release rate of 23.3 pg/(ml*min) was observed for the SE particles, a 47-fold increase in rate compared to neat CME. Moreover, a final release amount of 72.8±7% was detected for the SE particles, indicating an improvement of 68.6% in final released amount.

### Example 8

### In-Vivo Evaluation of Bioavailability after Oral Consumption

Phytocannabinoids serum concentrations were evaluated 90 min after oral administration to mice, as detailed above. The control group included a Cremophor EL (castor oil) based emulsion formulation, widely used for its potent solubilizing effect (Comelli F, Giagnoni G, Bettoni I, Colleoni M, 2008). The study group included the F2 tea formulation prepared at 0.62 mg CBD/mL, with both formulations providing 5mg/kg of CBD per body weight.

The bar graphs shown in FIG. 7 illustrate the relative amounts of the major phytocannabinoids (CBD, CBN, THC and CBC) that were quantified in the cannabis extract (MCE) used, as compared to the relative amounts of these components found in the blood serum in the experiment group (tea), and the control group (Cremophor EL). As shown in FIG. 7, both oral formulations were able to maintain the ratios between the extract phytocannabinoids components, indicating that the oral formulation allows for uniformity in the uptake of the various components. The measured serum concentrations of the four phytocannabinoids are shown in Table 3. The tea formulation exhibited somewhat lower serum concentrations compared to control group (not statistically viable). Cremophor is the benchmark in solubilizing lipophilic pharmaceutical ingredients (Gelderblom et al., 2001; Maincent and Zhang, 2016)

TABLE 1
CBDA, CBD, CBN, THC and CBC serum concentrations
(average ± standard error).

| | | CBD | CBN | THC | CBC |
|---|---|---|---|---|---|
| Control | Serum | 3.1 ± 0.97 | 0.1 ± 0.005 | 0.343 ± 0.09 | 0.247 ± 0.048 |
| group | concentration | | | | |
| | (μg/ml) | | | | |
| Experiment | Serum | 1.5 ± 0.13 | 0.125 ± 0.012 | 0.115 ± 0.015 | 0.210 ± 0.072 |
| Group | Concentration | | | | |
| | (μg/ml) | | | | |
| Prob > \|t\| | | 0.1 | 0.24 | 0.04 | 0.36 |

### Example 9

### Intestinal-Targeted Cannabis Delivery System

A pH-responsive delivery system was used for intestinal delivery of cannabis extract. The formulation composition included: (A) a whole-plant cannabis extract, (B) sodium alginate, (C) a lipid component, (triglycerides or fatty acids (glyceryl tristearate, oleic acid, linoleic acid)); and (D) a surfactant (methyl-β-cyclodextrin, β-cyclodextrin or lecithin). All formulations tested contain at least 40-50% w/w component A, 20-30% w/w B, 10-15% w/w C and 2-10% w/w D.

Formulation preparation: Whole-plant cannabis extract (A) and a lipid component (C) were placed in a closed vial and heated in a water bath at 80° C. Thereafter, hot water (80° C.) and a surfactant (D) were added to the vial and the mixture was vortexed and placed back in the water bath. At a different vial, a 2.5% w/v sodium alginate solution was prepared by dissolving sodium alginate in hot water (80° C.) under constant stirring for 1 h. After dissolving completely, the alginate solution was added to the first vial and the mixture was vortexed and heated repeatedly, then sonicated for 2 minutes using a probe sonicator to obtain a homogenous emulsion. Thereafter, the emulsion was added dropwise into CaCl₂ solution (0.7 M) and cannabis-loaded cross-linked alginate particles were formed. The particles were cured for 1 hour (h) in the same solution, removed by filtration, and washed with deionized water to remove excess CaCl2. The particles were then dried under vacuum at room temperature.

Whole-plant cannabis extract (100 mg) and glyceryl tristearate (25 mg) were placed in a closed vial and heated in a water bath at 80° C. until complete melting of the glyceryl tristearate. Then, hot water (80° C., 0.5 ml) and Methyl-β-cyclodextrin (5 mg) were added to the vial and the mixture was vortexed for 1 min and placed back in the water bath. At a different vial, sodium alginate (50 mg) was dissolved in 2 ml of hot water (80′C) under constant stirring for 1 h. When it dissolved completely, the alginate solution was added to the first vial and the mixture was vortexed and heated repeatedly, then sonicated for 2 min using a probe sonicator to obtain a homogenous emulsion. Then, the emulsion was added dropwise into CaCl₂ solution (0.7 M) and cannabis-loaded cross-linked alginate particles were formed. The particles were cured for 1 h in the same solution, removed by filtration, and washed with deionized water to remove excess CaCl₂. The particles were then dried under vacuum at room temperature.

### REFERENCES

Beal, J. E., Olson, R., Laubenstein, L., Morales, J.0., Bellman, P., Yangco, B., Lefkowitz, L., Plasse, T. F., Shepard, K. V, 1995. Dronainol as a Treatment for Anorexia Associated with Weight Loss in Patients with AIDS 1, 89-97.

Berman, J. S., Symonds, C., Birch, R., 2004. Efficacy of two cannabis based medicinal extracts for relief of central neuropathic pain from brachial plexus avulsion: results of a randomised controlled trial 112, 299-306. https://doi.org/10.1016/j.pain.2004.09.013

Berman, P., Futoran, K., Lewitus, G.M., Mukha, D., Benami, M., Shlomi, T., Meiri, D., 2018. A new ESI-LC / MS approach for comprehensive metabolic profiling of phytocannabinoids in Cannabis. Sci. Rep. 1-15.

Colizzi, M., Bhattacharyya, S., 2017. Does Cannabis Composition Matter ? Differential Effects of Delta-9-tetrahydrocannabinol and Cannabidiol on Human Cognition 62-74. https://doi.org/10.1007/s40429-017-0142-2.

Comelli F, Giagnoni G, Bettoni I, Colleoni M, C. B., 2008. Antihyperalgesic Effect of a Cannabis sativa Extract in a Rat Model of Neuropathic Pain: Mechanisms Involved. Phytother. Res. 22, 1017-1024. https://doi.org/10.1002/ptr

Cyclodextrin-enabled Cannabinoid Formulations, 2018. 32.

Do, J. M. M., Russo, E. B., Russo, E. B., 2016. Cannabis and Cannabis Extracts Cannabis and Cannabis Extracts : Greater Than the Sum of Their Parts ? 9775. https://doi.org/10.1300/J175v01n03

Ellis, R. J., Toperoff, W., Vaida, F., Brande, G. Van Den, Gonzales, J., Gouaux, B.,

Bentley, H., Atkinson, J. H., 2009. Smoked Medicinal Cannabis for Neuropathic Pain in HIV: A Randomized , Crossover Clinical Trial 672-680. https://doi.org/10.1038/npp.2008.120

Fahr, A., Liu, X., 2007. Expert Opinion on Drug Delivery Drug delivery strategies for poorly water-soluble drugs Drug delivery strategies for poorly water-soluble drugs. Expert Opin. Drug Deliv 4, 403-416. https://doi.org/10.1517/17425247.4.4.403

Garrettx, E. R., Hunt, C. A., 1974. Physicochemical Properties, Solubility , and Protein Binding of Delta-9-Tetrahydrocannabinol. J. Pharm. Sci. 63, 1056-1064. https://doi.org/10.1002/jps.2600630705

Gelderblom, H., Verweij, J., Nooter, K., Sparreboom, A., 2001. Cremophor EL: the drawbacks and advantages of vehicle selection for drug formulation 37, 1590-1598.

Grotenhermen, F., 2004. Cannabinoids for therapeutic use: Designing systems to increase efficacy and reliability. Am. J. Drug Deliv. 2, 229-240. https://doi.org/10.2165/00137696-200402040-00003

Hammersley, R., Jenkins, R., Reida, M., 2001. Cannabis use and social identity 9, 133-150.

Hill, K. P., 2015. Medical Marijuana for Treatment of Chronic Pain and Other Medical and Psychiatric Problems A Clinical Review. https://doi.org/10.1001/jama.2015.6199

Holdcroft, A., Maze, M., Sci, F. M., Dore, C., 2006. A Multicenter Dose-escalation Study of the Analgesic and Adverse Effects of an Oral Cannabis Extract ( Cannador) for Postoperative Pain Management 1040-1046.

Maincent, J., Zhang, F., 2016. Recent advances in abuse-deterrent technologies for the delivery of opioids. Int. J. Pharm. 510, 57-72. https://doi.org/10.1016/j.ijpharm, 2016. 06. 012.

Nakagawa, K., Iwamoto, S., Nakajima, M., Shono, A., Satoh, K., 2004. Microchannel emulsification using gelatin and surfactant-free coacervate microencapsulation. J. Colloid Interface Sci. 278, 198-205. https://doi.org/10.1016/j.jcis.2004.05.031 Pouton, C. W., 2006. Formulation of poorly water-soluble drugs for oral administration:

Physicochemical and physiological issues and the lipid formulation classification system. Eur. J. Pharm. Sci. 29, 278-287. https://doi.org/10.1016/j.ejps.2006.04.016 Prajapati, B. G., patel, M. M., 2007. Conventional and alternative pharmaceutical methods to improve oral bioavailability of lipophilic drugs. Asian Journal Pharmaceutics.

Shpigel, T., Uziel, A., Lewitus, D. Y., 2018. SPHRINT—Printing Drug Delivery

Microspheres from Polymeric Melts. Eur. J. Pharm. Biopharm. 127, 398-406. https://doi.org/10.1016/j.ejpb.2018.03.006

Singh, Y., Gopal, J., Raval, K., Ali, F., Chaurasia, M., Jain, N. K., Chourasia, M. K., 2017. Nanoemulsion : Concepts , development and applications in drug delivery. J. Control. Release 252, 28-Δ9.

Strasser, F., Luftner, D., Possinger, K., Ernst, G., Ruhstaller, T., Meissner, W., Ko, Y., Schnelle, M., Reif, M., Cerny, T., 2014. Journal of clinical oncology Comparison of Orally Administered Cannabis Extract and Delta-9-Tetrahydrocannabinol in Treating Patients With Cancer-Related Anorexia-Cachexia Syndrome : A Multicenter , Phase III , Randomized , Double-Blind , Placebo-Controlled Clinical Trial From the Cannabis-In-24. https://doi.org/10.1200/JCO.2005.05.1847
Sulak, D., Saneto, R., Goldstein, B., 2017. Epilepsy & Behavior The current status of artisanal cannabis for the treatment of epilepsy in the United States. Epilepsy Behay. 6-11. https://doi.org/10.1016/j.yebeh.2016.12.032
Tang, B., Cheng, G., Gu, J. C., Xu, C.H., 2008. Development of solid self-emulsifying drug delivery systems: preparation techniques and dosage forms. Drug Discov. Today 13, 606-612. https://doi.org/10.1016/j.drudis.2008.04.006
Thompson, A. J., Holmes, J. A., 2015. www.australianprescriber.com an independent review 38.
Todaro, B., York, N., 2012. Cannabinoids in the Treatment of Chemotherapy-Induced Nausea and Vomiting 10, 487-Δ92.
Zuardi, A. W., Moreira, F. A., Farmacologia, D., Neurologia, D., 2006. constituent , as an antipsychotic drug 39, 421-429.

1-40. (canceled)

41. A solid self-emulsifying Cannabis plant extract formulation comprising whole cannabis plant extract, a solid carrier and a lipidic component wherein upon rehydrating said solid formulation in liquid, the cannabis plant extract is self-emulsified within said liquid.

42. The formulation of claim 41, further comprising a solubility enhancer.

43. The formulation according to claim 41, further comprising a powdered form of non-dairy milk and/or of a plant-based protein.

44. The formulation according to claim 41, wherein the cannabis plant extract comprises about 30-60% w/w of said solid formulation.

45. The formulation according to claim 41, wherein said solid carrier comprises gelatin, carrageenan, alginate, or combinations thereof

46. The formulation according to claim 41, wherein said solid carrier comprises about 20-40% w/w of the solid formulation.

47. The formulation according to claim 41, wherein said lipid component comprises:
tristearin, Glyceryl tristearate, fatty acid, oleic acid, linoleic acid, or any combination thereof.

48. The formulation according to claim 41, wherein said lipid component comprises about 10-50% w/w of the solid formulation.

49. The formulation according to claim 42, wherein said solubility enhancer comprises a surfactant, selected from: Methylated-β-cyclodextrin (m-β-CD), β-cyclodextrin, lecithin, or combinations thereof.

50. The formulation according to claim 42, wherein said solubility enhancer comprises about 2-10% w/w of the solid formulation.

51. The formulation according to claim 43, wherein the powdered form of non-dairy milk comprises rice milk, soy milk, almond milk, oat milk, coconut milk hemp milk, or combinations thereof.

52. The formulation according to claim 43, wherein the powdered form of the plant-based proteins comprises soy protein, brown rice protein, pea protein, spirulina protein, hemp seed protein, flaxseed protein, pumpkin seed protein, sunflower seed protein chia seed protein, or combinations thereof.

53. The formulation according to claim 43, wherein the powdered form of non-dairy milk and/or plant-based protein comprises about 2-10% w/w of the solid formulation.

54. The formulation according to claim 41, wherein the liquid is warm, at a temperature of over about 50° C.

55. The formulation according to claim 41, wherein the liquid is water.

56. The formulation according to claim 41, wherein the solid self-emulsifying Cannabis plant extract formulation comprises whole cannabis plant extract, gelatin, and tristearin, wherein upon rehydrating said solid formulation in liquid, the cannabis plant extract is self-emulsified within said liquid.

57. The formulation according to claim 41, wherein the loading efficiency of the cannabis plant extract in the formulation is about 107±2.9% in the dry formulation and about 92±0.9% in the emulsion.

58. A method of preparing the solid self-emulsifying Cannabis plant extract formulation of claim 41, the method comprising:
a) heating the cannabis extract and a lipid or fatty acid in a suitable container to obtain a first mixture;
b) dissolving a solid carrier and emulsifying agent in heated aqueous solution to obtain a second mixture;
c) mixing the first mixture and the second mixture to obtain a mixture,
d) heating and/or mixing the mixture to obtain a homogeneous solution or emulsion; and
e) drying the homogenous solution or emulsion to obtain a solid mixture.

59. The method according to claim 58 further comprising a step of grinding the solid mixture to solid particles.

60. The method according to claim 58, wherein the first mixture is heated to a temperature of about 50-100° C.; and/or wherein the second mixture is dissolved in water at a temperature of about 40-95° C.
